(19) United States
(12) Patent Application Publication
(10) Pub. No.: US 2023/0149228 A1
(43) Pub. Date: May 18, 2023

(54) ABSORBENT LAYERS SUITABLE FOR USE IN ABSORBENT ARTICLES AND METHODS FOR MAKING SAME

(71) Applicant: Dow Global Technologies LLC, Midland, MI (US)

(72) Inventors: Eduardo Alvarez-Acedo, Tarragona (ES); Aleksandar Stoiljkovic, Waedenswil (CH); Barbara Bonavoglia, Horgen (CH); Edit Berczi, Horgen (CH)

(21) Appl. No.: 17/995,453

(22) PCT Filed: Apr. 12, 2021
(86) PCT No.: PCT/US2021/026855

§ 371 Date: Oct. 4, 2022

Related U.S. Application Data
(60) Provisional application No. 63/009,655, filed on Apr. 14, 2020.

(51) Int. Cl. A61F 13/539; A61F 13/15

(52) U.S. Cl. CPC A61F 13/539; A61F 13/15658; A61F 2013/53024

(57) ABSTRACT

Provided are absorbent layers and articles, and methods for making absorbent layers and articles. The absorbent layer is suitable for use in an absorbent article and may include a nonwoven that includes bicomponent fibers and superabsorbent polymer material interconnected within the nonwoven. The absorbent layer according to different embodiment may include a three-dimensional random loop material with superabsorbent polymer material adhered to the three-dimensional random loop material. Absorbent articles may comprise the absorbent layer according to embodiments of the present disclosure. The method for making the absorbent layer includes providing a nonwoven and dosing the nonwoven with superabsorbent polymer material.

### TECHNICAL FIELD

Embodiments of the present disclosure generally relate to absorbent layers and articles, and methods relating to making such absorbent layers and articles.

### INTRODUCTION

Absorbent articles often include different layers. For example, a conventional diaper may consist of a topsheet formed from a polypropylene nonwoven, a backsheet formed from a polyethylene film, an acquisition distribution layer (ADL) formed from a polyester nonwoven, and an absorbent core including equal amounts of superabsorbent polymer material (SAP) and cellulose fluff pulp. The cellulose fluff pulp in the absorbent core of a conventional diaper serves to hold the liquid insult while the SAP swell and absorbs the insult. The ADL acts to improve the rate of liquid uptake, distribution, and retention. There are several drawbacks, however, to including multiple or complex materials or layers, such as cellulose fluff pulp or an ADL. For example, in diapers, cellulose fluff pulp can contribute to sagging as well as a lack of extensibility and recyclability, and an ADL can significantly increase design costs and decrease comfort.

In recent years, absorbent articles, such as diapers, adult incontinence, and feminine hygiene products, have moved towards articles with enhanced comfort and simplified structure. For example, many diapers no longer include cellulose fluff pulp in the absorbent core and instead include a fluff-less alternative. However, a disadvantage of fluff-less designs for absorbent articles is the concern from end-purchasers who prefer to buy thicker, fluff-rich articles due to the perception of greater comfort or safety. Another disadvantage of many fluff-less designs is that, as a result of being thin, they can distribute liquid disproportionately throughout an absorbent article, which, for example, in diapers leads to risk of leaks and worse absorption performance management.

Accordingly, there remains a need for less costly and more efficiently produced absorbent layers and cores suitable for use in absorbent articles, and processes for making such absorbent layers and cores, that eliminate the need for use of layers or materials, such as cellulose fluff pulp and an ADL, while maintaining or improving other desirable properties, such as liquid absorbency, recyclability, extensibility, and comfort.

### SUMMARY

Embodiments of the present disclosure provide an absorbent layer suitable for use in an absorbent article that in some aspects reduces manufacturing costs, improves manufacturing efficiency, and delivers a unique combination of liquid absorbency, recyclability, extensibility, and comfort. For example, in some aspects, articles including the absorbent layer according to embodiments of the present disclosure not only eliminate the need for use of materials, such as cellulose pulp or an ADL, but also exhibit improved absorbency and extensibility properties. Such absorbent layers, in some aspects, can also advantageously help with recyclability and maintain an appearance of fluff without relying on additional materials, such as cellulose pulp.

Disclosed herein is an absorbent layer suitable for use in an absorbent article. In embodiments, the absorbent layer comprises a nonwoven comprising a plurality of bicomponent fibers, wherein each bicomponent fiber has a first region and a second region, wherein the weight ratio of the first region to the second region is at least 10/90 and not more than 90/10, wherein the first region comprises a first polymer composition in an amount of at least 75 wt. % based on total weight of the first region and the second region comprises a second polymer composition in an amount of at least 75 wt. % based on total weight of the second region; and a superabsorbent polymer material interconnected within the nonwoven. In embodiments, the nonwoven of the absorbent layer is a hydrophilic-treated nonwoven. In additional embodiments, the weight ratio of nonwoven to superabsorbent polymer material is at least 20/80 and not more than 80/20.

Also disclosed herein is a method for making an absorbent layer suitable for use in an absorbent article. In embodiments, the method comprises providing a nonwoven comprising a plurality of bicomponent fibers, wherein each bicomponent fiber has a first region and a second region; wherein the weight ratio of the first region to the second region is at least 10/90 and is not more than 90/10; wherein the first region comprises a first polymer composition in an amount of at least 75 wt. % based on total weight of the first region and the second region comprises a second polymer composition in an amount of at least 75 wt. % based on total weight of the second region; and dosing superabsorbent polymer material on the nonwoven such that the superabsorbent polymer material is interconnected within the nonwoven.

Also disclosed herein is an absorbent layer suitable for use in an absorbent article that comprises a three-dimensional random loop material. In embodiments, the absorbent layer comprises a three-dimensional random loop material; and a superabsorbent polymer material adhered to the three-dimensional random loop material.

Additional features and advantages of the embodiments will be set forth in the detailed description which follows, and in part will be readily apparent to those skilled in the art from that description or recognized by practicing the embodiments described herein, including the detailed description which follows, the claims, as well as the appended figures.

It is to be understood that both the foregoing and the following description describe various embodiments and are intended to provide an overview or framework for understanding the nature and character of the claimed subject matter.

### BRIEF DESCRIPTION OF THE DRAWINGS

FIG. 1 is a schematic illustrating an embodiment of the method disclosed herein.

FIG. 2 is another schematic illustrating another embodiment of the method described herein.

### DETAILED DESCRIPTION

Aspects of the disclosed absorbent layers, and methods for making absorbent layers, which may be used in absorbent articles, are described in more detail below. It is noted, however, that the below is merely an illustrative implementation of the aspects of the invention. The embodiments of the present invention are applicable to other technologies that are susceptible to similar problems as those discussed above. For example, although the disclosed absorbent layers are suitable for use in absorbent articles, such absorbent layers are not limited to use in absorbent articles, and may be used in the production of other articles.

As used herein, the term “interpolymer” refers to polymers prepared by the polymerization of at least two different types of monomers. The generic term interpolymer thus includes copolymers (employed to refer to polymers prepared from two different types of monomers), and polymers prepared from more than two different types of monomers.

As used herein, the term “polymer” means a polymeric compound prepared by polymerizing monomers, whether of the same or a different type. The generic term polymer thus embraces the term homopolymer (employed to refer to polymers prepared from only one type of monomer, with the understanding that trace amounts of impurities can be incorporated into the polymer structure), and the term interpolymer as defined herein. Trace amounts of impurities (for example, catalyst residues) may be incorporated into and/or within the polymer. A polymer may be a single polymer, a polymer blend or polymer mixture.

As used herein. the term “polyolefin” refers to a polymer that comprises, in polymerized form, a majority amount of olefin monomer, for example ethylene or propylene (based on the weight of the polymer), and optionally may comprise one or more comonomers

As used herein, the term “polyethylene” refers to polymers comprising greater than 50% by weight of units which are derived from ethylene monomer, and optionally, one or more comonomers. This may include polyethylene homopolymers or copolymers (meaning units derived from two or more comonomers). Common forms of polyethylene known in the art include Low Density Polyethylene (LDPE); Linear Low Density Polyethylene (LLDPE); Ultra Low Density Polyethylene (ULDPE); Very Low Density Polyethylene (VLDPE); single-site catalyzed Linear Low Density Polyethylene, including both linear and substantially linear low density resins (m-LLDPE); Medium Density Polyethylene (MDPE); and High Density Polyethylene (HDPE).

As used herein, the term “polypropylene” refers to polymers comprising greater than 50%, by weight, of units derived from propylene monomer, and optionally, one or more comonomers. This may include homopolymer polypropylene, random copolymer polypropylene, impact copolymer polypropylene, and propylene-based plastomers or elastomers (“PBE” or “PBPE”). PBE or PBPE polymers are further described in detail in the U.S. Pat. Nos. 6,960,635 and 6,525,157, incorporated herein by reference. Such polymers are commercially available from The Dow Chemical Company, under the tradename VERSIFY™ or from ExxonMobil Chemical Company, under the tradename VISTAMAXX™.

As used herein, the term “polyethylene terephthalate” (PET) refers to a polyester formed by the condensation of ethylene glycol and terephthalic acid.

As used herein, the terms “nonwoven,” “nonwoven web,” and “nonwoven fabric” are used herein interchangeably. “Nonwoven” refers to a web or fabric having a structure of individual fibers or threads which are randomly interlaid, but not in an identifiable manner as is the case for a knitted fabric.

As used herein, the term “meltblown” refers to the fabrication of nonwoven fabrics via a process which generally includes the following steps: (a) extruding molten thermoplastic strands from a spinneret; (b) simultaneously quenching and attenuating the polymer stream immediately below the spinneret using streams of high velocity heated air; (c) collecting the drawn strands into a web on a collecting surface. Meltblown webs can be bonded by a variety of means including, but not limited to, autogeneous bonding, i.e., self bonding without further treatment, thermo-calendaring process, adhesive bonding process, hot air bonding process, needle punch process, hydroentangling process, and combinations thereof.

As used herein, the term “spunbond” refers to the fabrication of nonwoven fabric including the following steps: (a) extruding molten thermoplastic strands from a plurality of fine capillaries called a spinneret; (b) quenching the strands with a flow of air which is generally cooled in order to hasten the solidification of the molten strands; (c) attenuating the stands by advancing them through the quench zone with a draw tension that can be applied by either pneumatically entraining the stands in an air stream or by winding them around mechanical draw rolls of the type commonly used in the textile fibers industry; (d) collecting the drawn strands into a web on a foraminous surface, e.g., moving screen or porous belt; and (e) bonding the web of loose strands into a nonwoven fabric. Bonding can be achieved by a variety of means including, but not limited to, thermo-calendaring process, adhesive bonding process, hot air bonding process, needle punch process, hydroentangling process, and combinations thereof.

As used herein, the term “dosing superabsorbent polymer material” refers to depositing an amount of superabsorbent polymer material onto or within the matrix of the nonwoven such that the superabsorbent polymer material is not agglomerated on or within the nonwoven.

As used herein, the term “interconnected within the nonwoven” refers to when superabsorbent polymer material is affixed in a stable manner onto or within the matrix of a nonwoven such that the superabsorbent polymer material is not agglomerated on or within the nonwoven and is not easily displaced from the nonwoven or an absorbent layer, core, or article comprising the nonwoven.

### Absorbent Layer—Nonwoven and Bicomponent Fibers

An absorbent layer comprises a nonwoven comprising a plurality of bicomponent fibers as discussed herein.

The nonwoven comprising the plurality of bicomponent fibers according to the present disclosure can be produced via different techniques. Such techniques for forming a nonwoven from bicomponent fibers include melt spinning, melt blown process, spunbond process, staple process, carded web process, air laid process, thermo-calendering process, adhesive bonding process, hot air bonding process, needle punch process, hydroentangling process, and electrospinning process. For example, the bicomponent fibers can be processed directly into a planar sheet-like fabric structure and then bonded chemically, thermally and/or interlocked mechanically to achieve a cohesive nonwoven. The nonwoven of the present disclosure can be formed by any method known in the art, such as those mentioned above.

In some embodiments, the nonwoven comprising the plurality of bicomponent fibers has a basis weight in the range of from 50 to 500 grams per square meter (gsm). All individual values and subranges of from 50 to 500 gsm are included herein and disclosed herein; for example, the nonwoven can be from a lower limit of 50, 100, 150, 200, 250, 300, 350, 400, or 450 gsm to an upper limit of 100, 150, 200, 250, 300, 350, 400, 450, or 500 gsm.

In some embodiments, the nonwoven comprising the plurality of bicomponent fibers has a thickness of at least 10 micron/gsm, where thickness is measured according to EDANA 120.6 and basis weight is measured according to EDANA 130.1. All individual values and subranges of at least 10 microns/gsm are included and disclosed herein; for example, the nonwoven comprising the plurality of bicomponent fibers can have a thickness of at least 10 micron/gsm, 12 micron/gsm, or 14 micron/gsm, or 16 micron/gsm and can have a thickness in the range from 10 micron/gsm to 60 micron/gsm, 10 micron/gsm to 40 micron/gsm, 10 micron/gsm to 20 micron/gsm, 15 micron/gsm to 60 micron/gsm, 15 micron/gsm to 40 micron/gsm, or 15 micron/gsm to 20 micron/gsm, where thickness is measured according to EDANA 120.6 and basis weight is measured according to EDANA 130.1.

With respect to bicomponent fibers, bicomponent fibers are filaments made up of two different regions comprising different polymer compositions that are extruded from the same spinneret with both compositions contained within the same filament. When the filament leaves the spinneret, it consists of non-mixed components that are fused at the interface. The two regions may differ in their chemical and/or physical properties, which allows the bicomponent fiber to meet a wider variety of desired properties as the functional properties of the polymer compositions can be joined into one filament. The bicomponent fibers according to embodiments of the present disclosure can be formed by any conventional spinning technique known in the art including melt spinning. In melt spinning, the two different polymer compositions are melted, coextruded and forced through the fine orifices in a metallic plate called spinneret into air or other gas, where they are cooled and solidified forming bicomponent fibers. The solidified bicomponent fibers may be drawn off via air jets, rotating rolls, or godets, and can be laid on a conveyer belt as a web for forming the nonwoven.

The bicomponent fibers according to embodiments of the present disclosure can contain two regions in a variety of different configurations. Examples of bicomponent fiber configurations are core-sheath, side-by-side, segmented pie, or islands-in-the-sea. In some embodiments, the bicomponent fibers may have a core-sheath configuration wherein a cross section of the fiber shows one region, a core, surrounded by another region, a sheath. In other embodiments, the bicomponent fibers may have a side-by-side configuration. In further embodiments, the bicomponent fibers may have a segmented pie configuration wherein a cross section of the fiber shows one region occupying a portion, for example a quarter, a third, a half of the cross section and a second region occupies the remainder of the cross section. Those skilled in the art will appreciate that the composition and configuration of the bicomponent fiber according to embodiments of the present invention assists with the loft of the nonwoven and/or curl of the fibers, which promotes space for liquid absorbency and swelling of the superabsorbent polymer material described below.

In some embodiments, the bicomponent fibers according to the present disclosure have a fiber diameter in the range of from 5 to 100 micrometers. All individual values and subranges of from 5 to 100 micrometers are included herein and disclosed herein; for example, the fiber diameter of the bicomponent fibers can be from a lower limit of 5, 10, 15, 20, 25, 30, 35, 40, 45, 50, 55, 60, 75, 80, 85, 90, or 95 micrometers to an upper limit of 10, 15, 20, 25, 30, 35, 40, 45, 50, 55, 60, 75, 80, 85, 90, 95, or 100 micrometers.

The bicomponent fibers each have a first region and a second region. The regions of the bicomponent fibers relate to the compositions that are extruded from the spinneret. For example, in a core-sheath configuration, the first region can be the core and the second region can be the sheath.

In some embodiments, the weight ratio of the first region to the second region is at least 10/90, or 20/80 or 30/70 or 40/60 and is not more than 90/10 or 80/20 or 70/30 or 60/40. For example, in some embodiments, the weight ratio of the first region to the second region is at least 10/90 and not more than 90/10; at least 20/80 and not more than 80/20; at least 70/30 and not more than 70/30; or at least 40/60 and not more than 60/40.

The first region comprises a first polymer composition in an amount of at least 75 wt. % based on total weight of the first region. In some embodiments, the first polymer composition can comprise from 75 to 100 wt. % of the total weight of the first region. All individual values and subranges of at least 75 wt. % are included herein and disclosed herein; for example, the first polymer composition can be from a lower limit of 75, 80, 85, 90, or 95 wt. % to an upper limit of 80, 85, 90, 95, 100 wt. % based on total weight of the first region.

The first polymer composition according to embodiments of the present disclosure comprises one or more of the following: a polypropylene, a polyethylene, a polyethylene terephthalate, or a combination or blend thereof. In some embodiments, the first polymer composition may further comprise additional components, such as, one or more other polymers and/or one or more additives. Such additives include, but are not limited to, antistatic agents, color enhancers, dyes, lubricants, fillers, pigments, primary antioxidants, secondary antioxidants, processing aids, UV stabilizers, anti-blocks, slip agents, tackifiers, fire retardants, anti-microbial agents, odor reducer agents, anti-fungal agents, and combinations thereof. Effective amounts of additives are known in the art and depend on parameters of the polymers in the composition and conditions to which they are exposed.

In some embodiments, the first region further comprises a polyolefin elastomer. For example, a polyolefin elastomer may be provided to improve the extensibility of the nonwoven and absorbent layer. In some embodiments, the polyolefin elastomer can be a block copolymer. In some embodiments where polyolefin elastomer is used in the first region, the first region can comprise 25 wt. % or less of the polyolefin elastomer based on the total weight of the first region. Examples of commercially available polyolefin elastomers that can be used include polyolefin elastomers available from The Dow Chemical Company under the names VERSIFY™ ENGAGE™, AFFINITY™, and INFUSE™, polyolefin elastomers available from ExxonMobil Chemical Co. under the name VISTAMAXX™, and polyolefin elastomers available from Idemitsu under the name L-MODU™. The first region can be prepared from the components discussed above using techniques known to those of skill in the art based on the teachings herein.

The second region comprises a second polymer composition in an amount of at least 75 wt. % based on total weight of the second region. In some embodiments, the second polymer composition can comprise from 75 to 100 wt. % of the total weight of the second region. All individual values and subranges of at least 75 wt. % are included herein and disclosed herein; for example, the second polymer composition can be from a lower limit of 75, 80, 85, 90, or 95 wt. % to an upper limit of 80, 85, 90, 95, 100 wt. % based on total weight of the second region.

The second polymer composition according to embodiments of the present disclosure comprises one or more of the following: a polypropylene, a polyethylene, a polyethylene terephthalate, or a combination or blend thereof. In some embodiments, the second region and/or second polymer composition may further comprise additional components, such as, one or more other polymers and/or one or more additives. Such additives include, but are not limited to, antistatic agents, color enhancers, dyes, lubricants, fillers, pigments, primary antioxidants, secondary antioxidants, processing aids, UV stabilizers, anti-blocks, slip agents, tackifiers, fire retardants, anti-microbial agents, odor reducer agents, anti-fungal agents, and combinations thereof. Effective amounts of additives are known in the art and depend on parameters of the polymers in the composition and conditions to which they are exposed.

In some embodiments, the second region may comprise a polyolefin elastomer. For example, polyolefin elastomer may be provided to increase the extensibility of the absorbent layer. In some embodiments, the polyolefin elastomer can be a block copolymer. In some embodiments where polyolefin elastomer is used in the second region, the second region can comprise 25 wt. % or less of the polyolefin elastomer based on the total weight of the second region. Examples of commercially available polyolefin elastomers that can be used include polyolefin elastomers available from The Dow Chemical Company under the names VERSIFY™ ENGAGE™, AFFINITY™, and INFUSE™, polyolefin elastomers available from ExxonMobil Chemical Co. under the name VISTAMAXXT™, and polyolefin elastomers available from Idemitsu under the name L-MODUT™. The second region can be prepared from the components discussed above using techniques known to those of skill in the art based on the teachings herein.

Each bicomponent fiber has a centroid and each region of the bicomponent fiber has its own centroid. As used herein centroid means the arithmetic mean of all the points of the region of a cross-section of the fiber or a specific region of the fiber. For example, the bicomponent fiber according to embodiments of the present disclosure has a fiber centroid, which can be designated as C_{f}, and a region of the bicomponent fiber (e.g., the first or second region) has an independent centroid, which can be designated as Cᵣₓ, where x is a designation of the region (e.g., the first region can be designated as Cᵣ₁ and the second region can be designates as Cᵣ₂), and where “r” is the average distance from C_{f} to the outer surface of the bicomponent fiber and is calculated as \sqrt{A/π}, where A is the area of the bicomponent fiber cross-section. For a concentric core-sheath bicomponent fiber configuration, the core and sheath have the same centroid. For an eccentric core-sheath bicomponent fiber configuration, the core and the sheath have different centroids. According to one embodiment, the first region and the second region have centroids that are different from the centroid of the fiber. According to another embodiment at least one of the regions has a centroid that is different from the centroid of the fiber. According to another embodiment the fibers have substantially a concentric core-sheath configuration.

In addition, in embodiments where the bicomponent fibers have a core-sheath configuration, each bicomponent fiber can have a concentricity value. Concentricity can be calculated by the formula C=Wₘᵢₙ/Wₘₐₓ×100%, where C equals the concentricity value expressed as a percentage, Wₘᵢₙ is a measurement based on a cross section of the fiber of the minimum or shortest distance from the outer surface of the fiber to the outer portion of the core region of the fiber, and Wₘₐₓ is a measurement based on a cross section of the fiber of the maximum or longest distance from the outer surface of the fiber to the outer portion of the core region of the fiber. For example, in a concentric core-sheath configuration, Wₘᵢₙ equals Wₘₐₓ because the distance from the outer surface of the bicomponent fiber to the core region is the same and symmetric over the entire cross section of the fiber, and so a bicomponent fiber having a concentric core-sheath configuration has a concentricity value of 100%. As another example, in an eccentric core-sheath configuration, the Wₘᵢₙ and Wₘᵢₙ are different, and Wₘᵢₙ can be, for example, 5 micrometers and Wₘₐₓ can be, for example, 6 micrometers, which results in a concentricity value of 83.33%. In embodiments, the bicomponent fibers disclosed herein can have a concentricity of from 60% to 100%, from 70% to 100%, 80% to 100%, 90% to 100%, or 95% to 100%.

As discussed above, a primary disadvantage of incumbent fluff-less designs for absorbent articles is the concern from end-purchasers, who often prefer to buy thicker, fluff-rich article due to the perception of higher absorption, comfort, or safety. In addition, the distribution of liquid within a fluff-less designs is often not homogeneous. Without being bound by theory, it is believed that the nonwoven comprising a plurality of bicomponent fibers according to embodiments of the present disclosure may provide fluff and adequate space for superabsorbent polymer material to swell and absorb liquid without the need of other materials, such as an acquisition distribution layer or cellulose pulp. The nonwoven has fluff when formed from embodiments of the bicomponent fibers disclosed herein and can be compressed and extended while retaining its shape.

### Absorbent Layer—Three-Dimensional Random Loop Material

In different embodiments, an absorbent layer suitable for use in an absorbent article can comprise a three-dimensional random loop material (also referred to as a “3DRLM”) as opposed to or in addition to the nonwoven comprising a plurality of bicomponent fibers described above. A 3DRLM is a mass or a structure of a multitude of loops formed by allowing continuous fibers to wind permitting respective loops to come in contact with one another in a molten state and to be heat-bonded, or otherwise melt-bonded, at most of the contact points. These 3DRLMs are further described in detail in WO 2018/236545, which is incorporated herein by reference.

A nonlimiting method for producing 3DRLM includes the steps of (a) heating a molten olefin-based polymer, at a temperature 10° C.-140° C. higher than the melting point of the polymer in a typical melt-extruder; and (b) discharging the molten polymer to the downward direction from a nozzle with plural orifices to form loops by allowing the fibers to fall naturally (due to gravity). The polymer may be used in combination with a thermoplastic elastomer, thermoplastic non-elastic polymer or a combination thereof. The distance between the nozzle surface and take-off conveyors installed on a cooling unit for solidifying the fibers, melt viscosity of the polymer, diameter of orifice and the amount to be discharged are the elements which decide loop diameter and fineness of the fibers. Loops are formed by holding and allowing the delivered molten fibers to reside between a pair of take-off conveyors (belts, or rollers) set on a cooling unit (the distance therebetween being adjustable), bringing the loops thus formed into contact with one another by adjusting the distance between the orifices to this end such that the loops in contact are heat-bonded, or otherwise melt-bonded, as they form a three-dimensional random loop structure. Then, the continuous fibers, wherein contact points have been heat-bonded as the loops form a three-dimensional random loop structure, are continuously taken into a cooling unit for solidification to give a net structure. Thereafter, the structure is cut into a desired length and shape such that it is suitable for use as part of an absorbent layer.

Properties, such as, the loop diameter and fineness of the fibers constituting the cushioning net structure depend on the distance between the nozzle surface and the take-off conveyor installed on a cooling unit for solidifying the polymer, melt viscosity of the polymer, diameter of orifice and the amount of the polymer to be delivered therefrom. For example, a decreased amount of the polymer to be delivered and a lower melt viscosity upon delivery result in smaller fineness of the fibers and smaller average loop diameter of the random loop. On the contrary, a shortened distance between the nozzle surface and the take-off conveyor installed on the cooling unit for solidifying the polymer results in a slightly greater fineness of the fiber and a greater average loop diameter of the random loop. These conditions in combination afford the desirable fineness of the continuous fibers of from 100 denier to 100000 denier and an average diameter of the random loop of not more than 100 millimeter (mm), or from 1 mm, or 2 mm, or 10 mm to 25 mm, or 50 mm. By adjusting the distance to the aforementioned conveyor, the thickness of the structure can be controlled while the heat-bonded net structure is in a molten state and a structure having a desirable thickness and flat surface formed by the conveyors can be obtained. Too great a conveyor speed results in failure to heat-bond the contact points, since cooling proceeds before the heat-bonding. On the other hand, too slow a speed can cause higher density resulting from excessively long dwelling of the molten material.

As discussed above, a primary disadvantage of incumbent fluff-less designs for absorbent articles is the concern from end-purchasers, who often prefer to buy thicker, fluff-rich articles due to the perception of higher absorption, comfort, or safety. In addition, the distribution of liquid within a fluff-less articles is often not homogeneous. Without being bound by theory, it is believed that the 3DRLM according to embodiments of the present disclosure may provide a support structure and absorb stress for use as part of an absorbent article. The 3DRLM can be formed into a three-dimensional geometric shape to form a sheet and can be an elastic material which can be compressed and stretched while returning to its original geometric shape.

The 3DRLM comprises one or more polymers, including, for example, a polypropylene, a polyethylene, a polyethylene terephthalate, or a combination or blend thereof. In some embodiments, the 3DRLM can replace the nonwoven discussed above such that the absorbent layer comprises a 3DRLM. In other embodiments, the 3DRLM can be used in addition with the nonwoven for forming the absorbent layer suitable for use in an absorbent article.

### Absorbent Layer—Superabsorbent Polymer (SAP)

An absorbent layer suitable for use in an absorbent article according to embodiments of the present disclosure comprises a superabsorbent polymer material as described herein.

As used herein, the term “superabsorbent polymer material” refers to water-swellable, substantially water insoluble material that is capable of absorbing at least 10 times its weight in an aqueous solution containing 0.9 weight percent sodium chloride. The use of superabsorbent polymer material in absorbent articles for facilitating the absorption of liquid is well known. The specific type of superabsorbent polymer material according to embodiments of the present disclosure may be in any form which is suitable for use in absorbent articles including, for example, particles, fibers, flakes, cubes, and spheres. Examples of organic materials suitable for use as superabsorbent polymer material can include synthetic materials such as synthetic hydrogel polymers and natural materials such as polysaccharides and polypeptides. Other suitable materials include hydrolyzed acrylonitrile grafted starch, acrylic acid grafted starch, and isobutylene maleic anhydride copolymers and mixtures thereof. Superabsorbent polymer material can be surface cross-linked so that the outer surface of the superabsorbent polymer material has a higher crosslink density than the inner part of the superabsorbent polymer material.

In some embodiments, the weight ratio of the nonwoven to the superabsorbent polymer material is at least 20/80 or 30/70 or 40/60 and is not more than 80/20 or 70/30 or 60/40. For examples, in some embodiments, the weight ratio of the nonwoven to the superabsorbent polymer material is at least 20/80 and not more than 80/20, is at least 30/70 and not more than 70/30, is at least 40/60 and not more than 60/40. In other embodiments, where the absorbent layer comprises a 3DRLM in place of the nonwoven, the weight ration of the 3DRLM to the superabsorbent polymer material is at least 20/80 or 30/70 or 40/60 and is not more than 80/20 or 70/30 or 60/40. For examples, in some embodiments, the weight ratio of the 3DRLM to the superabsorbent polymer material is at least 20/80 and not more than 80/20, is at least 30/70 and not more than 70/30, is at least 40/60 and not more than 60/40.

In embodiments, the superabsorbent material is interconnected within the nonwoven. The superabsorbent material can be interconnected within the nonwoven by using different techniques. Such techniques for interconnecting the superabsorbent material within the nonwoven include use of an alternating electrical field, mechanical vibration system, ultrasonic bonding system, and dry impregnation methods. Without being bound by theory, it is believed that the nonwoven comprising a plurality of bicomponent fibers according to embodiments of the present disclosure provides space for the superabsorbent polymer material to infiltrate the nonwoven matrix to become interconnected within the nonwoven while the nonwoven has sufficient thickness to provide fluff and allow the superabsorbent polymer material to swell.

In other embodiments, where the absorbent layer comprises a 3DRLM, the superabsorbent polymer material is adhered to the 3DRLM. The superabsorbent material can be adhered to the 3DRLM by using different techniques, such as use of an adhesive, binder, or glue. In embodiments, the 3DRLM can comprise polymers that promote adhesion, such as ethylene acrylic acid copolymers, that can help adhere the superabsorbent polymer material to the 3DRLM. In other embodiments, an adhesive can be used for adhering the superabsorbent polymer material to the 3DRLM. Such an adhesive can be a solventless adhesive, a waterborne adhesive, or a solventborn adhesive.

In some embodiments, the nonwoven can be hydrophilically treated prior to adding and interconnecting the superabsorbent polymer material within the nonwoven so that the absorbent layer comprises a hydrophilic-treated nonwoven. In other embodiments, the superabsorbent polymer material can also be hydrophilically treated while being interconnected within the nonwoven. Those skilled in the art will appreciate that if the hydrophilic treatment is applied to the superabsorbent polymer material, the treatment should be applied to reduce or minimize its impact on the superabsorbent polymer material. The hydrophilic treatment can be applied via different techniques known in the art. Such techniques include corona or plasma treatment as well as solution spraying, spinning, coating, or the addition of hydrophilic additives into the nonwoven matrix. For example, in some embodiments, a hydrophilic-treated can be applied via a plasma treatment where the nonwoven is exposed to an atmospheric plasma comprising an inert gas and a substance having a polar group and which can be vaporized or made into an aerosol and which forms a free radical upon exposure to a dielectric barrier discharge. Atmospheric plasma systems and methods are generally described in U.S. Pat. No. 5,433,786, the disclosure of which is incorporated herein by reference.

To those skilled in the art, the nonwoven according to embodiments of the present disclosure affixes, without agglomeration, the superabsorbent polymer material and enhances liquid absorbency by providing room for the superabsorbent polymer material to swell during the absorption process. The configuration and arrangement of the nonwoven and superabsorbent polymer material in some aspects improve liquid absorption and enhance body comfort, cushioning effect, and extensibility without an increase in sagging and without the need for other materials, such as an acquisition distribution layer or cellulose fluff pulp. Likewise, according to other embodiments of the present disclosure, when the nonwoven is replaced by the 3DRLM, the 3DRLM serves, in part, the same function as a vessel for the superabsorbent polymer material and enhancer of properties such as comfort, cushioning, and extensibility without, for example, sagging, and without the need for other materials, such as an acquisition distribution layer or cellulose fluff pulp.

### Other Layers—Nonwoven Layer(s)

In some embodiments, an absorbent layer as described above can be bonded or adhered to other layers to form an absorbent core or article. For example, in some embodiments, the absorbent layer comprising the nonwoven can additionally be bonded to one or more nonwoven layers comprising one or more of the following: a polypropylene, a polyethylene, a polyethylene terephthalate, or a combination or blend thereof. In such an embodiment, the one or more nonwoven layers can be formed from a monofilament or formed from bicomponent fibers, such as the same or similar bicomponent fibers used to form the nonwoven. The one or more nonwoven layers can bond or adhere to the absorbent layer and act as a sheet or layer to further prevent the superabsorbent polymer material interconnected within the nonwoven (or adhered to the 3DRLM) of the absorbent layer from being dispersed and contaminating the environment. In some embodiments, the one or more nonwoven layers can be initially part of a prepared nonwoven roll which is unwound onto a belt such that the absorbent layer can be added to the prepared nonwoven roll during the manufacturing process.

In some embodiments, the one or more nonwoven layers can be hydrophilically treated. In some embodiments, superabsorbent polymer material can be interconnected within the one or more nonwoven layers.

### Absorbent Articles

The absorbent layer of the present disclosure can be incorporated in absorbent articles. The absorbent layer of the present disclosure is particularly useful in absorbent articles where extensibility, recyclability, and/or liquid absorbency is a desirable feature. The absorbent article will include at least one absorbent layer according to embodiments of the present disclosure and can include a number of other layers, such as the one or more nonwoven layers described above, as will be apparent to those of skill in the art based on the teaching herein. In embodiments, the absorbent article includes an absorbent layer of the present disclosure and is free from an acquisition distribution layer. In further embodiments, the absorbent article includes an absorbent layer of the present disclosure and is free from cellulose fluff pulp. In even further embodiments, the absorbent article includes an absorbent layer of the present disclosure and is free from cellulose fluff pulp and an acquisition distribution layer.

In some embodiments, an absorbent article can comprise two or more of the absorbent layers according to embodiments of the present disclosure. As another example, an absorbent article can include one absorbent layer of the present disclosure that is between two nonwoven layers that have the same composition as each other. As yet another example, an absorbent article can include one absorbent layer that is between two nonwoven layers that have different compositions.

For example, in one embodiment, an absorbent article can have an A/A structure, where “A” is the absorbent layer according to an embodiment of the present disclosure that comprises a hydrophilic-treated nonwoven and a superabsorbent polymer material interconnected within the nonwoven.

As another example, an absorbent article of the present disclosure can have a B/A/B structure, where “B” is a nonwoven layer comprising bicomponent fibers in a concentric core-sheath configuration, and where “A” is the absorbent layer described in the prior example.

As another example, an absorbent article of the present disclosure can have a B/A/C structure, where “B” is the nonwoven layer described in the prior example, where “A” is the absorbent layer described in the prior two examples, and where “C” is a different nonwoven layer comprising a hydrophilic-treated nonwoven that comprises bicomponent fibers in an eccentric core-sheath configuration.

Absorbent articles of the present disclosure can exhibit one or more desirable properties. For example, in some embodiments, absorbent articles can exhibit desirable properties such as improved liquid absorbency, extensibility, recyclability, and others while being free of other materials, such as cellulose fiber or an ADL.

The absorbent layers of the present disclosure can be used to form a variety of absorbent articles, including diapers, using techniques known to those of skill in the art. For example, in some embodiments, the absorbent layer of the present disclosure can be combined with and placed in between a liquid impermeable backsheet and a liquid permeable topsheet. The topsheet and backsheet could be made from any suitable material known to those skilled in the art, including, for example, a nonwoven. The absorbent article may also include other features known to those skilled in the art, including side panels, ears, leg cuffs, or a belt.

For example, in one embodiment, an absorbent article can comprise a E/B/A/C/D structure, where “E” is a liquid impermeable backsheet, “B” is a first nonwoven layer comprising monocomponent or bicomponent fibers, “A” is the absorbent layer according to embodiments of the present disclosure, “C” is a second nonwoven layer comprising bicomponent fibers having a side-by-side or eccentric core-sheath configuration, and “D” is a liquid permeable topsheet.

Absorbent articles that can be formed include, for example, diapers, face masks, wipes, tissues, feminine hygiene, and adult incontinence products.

### Methods for Making Absorbent Layers and Articles

A method for making an absorbent layer according to embodiments of the present disclosure comprises providing a nonwoven comprising a plurality of bicomponent fibers, wherein each bicomponent fiber has a first region and a second region; wherein the weight ratio of the first region to the second region is at least 10/90 and is not more than 90/10; wherein the first region comprises a first polymer composition in an amount of at least 75 wt. % based on total weight of the first region and the second region comprises a second polymer composition in an amount of at least 75 wt. % based on total weight of the second region; and dosing superabsorbent polymer material on the nonwoven such that the superabsorbent polymer material is interconnected within the nonwoven. The nonwoven can be formed using techniques described above and known to those of skill in the art based on the teaching herein, including use of meltblown or spunbond processes. The dosing of the superabsorbent polymer material can be accomplished using techniques described above and known to those of skill in the art based on the teaching herein. In embodiments, the superabsorbent polymer material is homogenously dosed onto the nonwoven so that the superabsorbent polymer material is distributed evenly. Such an embodiment can improve the liquid absorption process by distributing the swelling of the superabsorbent polymer material evenly throughout the absorbent layer.

In some embodiments, the method for making the absorbent layer can further comprise hydrophilically treating the nonwoven. The nonwoven can be hydrophilically treated using techniques described above and known to those of skill in the art based on the teaching herein, including corona and/or plasma treatment. In some embodiments, the method for making the absorbent layer can further comprise applying hot air after hydrophilically treating the nonwoven. Hot air can be used to assist in bonding the fibers and strengthening the nonwoven, and it can, in embodiments where moisture is added during the hydrophilic treatment process, assist in removing any moisture or water from the structure.

In some embodiments, a method for making an absorbent article can comprise providing a nonwoven layer so as to form multiple nonwoven layers. The nonwoven layer, in some embodiments, can have the same compositions as the nonwoven. In other embodiments, the nonwoven layer can include a different composition(s), than the nonwoven, known to those of skill in the art based on the teachings herein. In some embodiments, the nonwoven layer can be part of a prepared nonwoven roll which is unwound onto a manufacturing belt. In some embodiments, the method for making the absorbent article can further comprise placing the nonwoven onto the nonwoven layer and the dosing the nonwoven with the superabsorbent polymer material such that the superabsorbent polymer material is interconnected within the nonwoven. The nonwoven layer, in such an embodiment, can act as a layer to prevent the superabsorbent polymer material from being dispersed into the surrounding environment. In such an embodiment, the nonwoven layer and the nonwoven can be hydrophilically treated together, prior to dosing superabsorbent polymer material on the nonwoven.

In some embodiments, the method for making an absorbent article can comprise providing a first nonwoven layer; placing a nonwoven according to embodiments of the present disclosure on top of the first nonwoven layer; hydrophilically treating the first nonwoven layer and the nonwoven; dosing superabsorbent polymer material onto the nonwoven such that the superabsorbent polymer material is interconnected within the nonwoven to form an absorbent layer; applying hot air to the first nonwoven layer and the absorbent layer; providing a second nonwoven layer; placing the second nonwoven layer on top of the absorbent layer; and hydrophilically treating the combination of the first nonwoven layer, the absorbent layer, and the second nonwoven layer. In such an embodiment, the second nonwoven layer can have the same composition as the nonwoven of the absorbent layer or the second nonwoven layer can have a different composition. In such an embodiment, the method for making the absorbent article can further comprise applying hot air, after hydrophilically treating the second nonwoven layer, to remove any moisture or water. In such an embodiment, the method for making the absorbent article can result in an absorbent article having a B/A/C structure, as described above, where “B” is a first nonwoven layer, “A” is the absorbent layer comprising the nonwoven with superabsorbent polymer material interconnecting within, and “C” is a second nonwoven layer.

In some embodiments, the method for making the absorbent article can comprise providing a nonwoven according to embodiments of the present disclosure; hydrophilically treating the nonwoven; applying hot air to the nonwoven; providing a nonwoven layer; hydrophilically treating the nonwoven layer; applying hot air to the to the nonwoven layer; and dosing superabsorbent polymer material onto the nonwoven and the nonwoven layer such that the superabsorbent polymer material is interconnected within the nonwoven and the nonwoven layer.

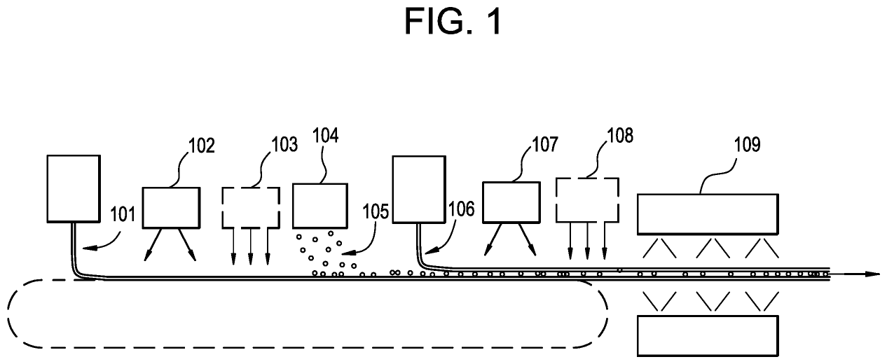

Referring now generally to FIG. 1, an illustration of an embodiment of a method for making an absorbent article as described herein is shown. The method disclosed herein can include providing a spunbond nonwoven 101. The nonwoven 101 comprises a plurality of bicomponent fibers according to embodiments of the present disclosure. Hot air 102 can be applied to assist with bonding or strengthening the nonwoven. The nonwoven 101 can receive a plasma and/or corona treatment 103 to create a hydrophilic-treated nonwoven. Superabsorbent polymer material 105 can then be dosed onto the nonwoven via a dosing and distribution system 104 that deposits a predetermined amount of superabsorbent polymer material homogenously onto the nonwoven 101 such that the superabsorbent polymer material 105 is interconnected within the nonwoven. Then, a spunbond nonwoven layer 106 can be placed onto the nonwoven 101. Hot air 107 can be applied to assist with bonding or strengthening the nonwoven layer. The nonwoven and the nonwoven layer can receive a plasma and/or corona treatment 108 to create a hydrophilic nonwoven layer and further hydrophilically treat the nonwoven. An absorbent article comprising the nonwoven 101 and the nonwoven layer 106 can result and can be placed through a hot air oven 109, where thereafter the absorbent article can be wound, slit, bonded, and/or combined with other components to form, for example, a diaper.

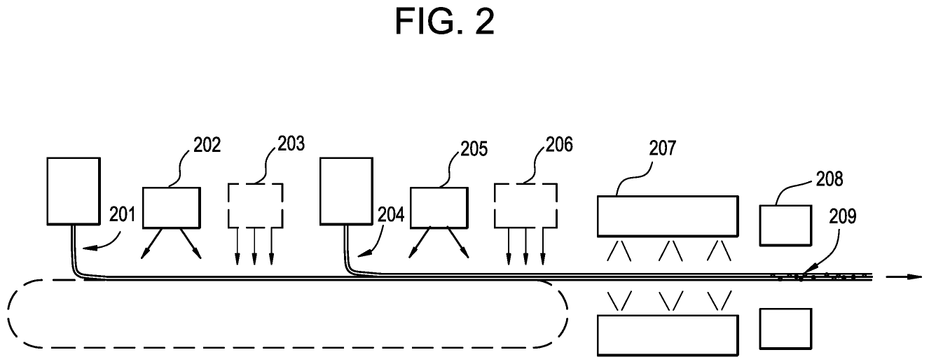

Referring now generally to FIG. 2, another illustration of an embodiment of a method for making an absorbent article as described herein is shown. The method disclosed herein can include providing a meltblown nonwoven layer 201; applying hot air 202 to the nonwoven; applying a hydrophilic treatment 203 such as a plasma and/or corona treatment to the nonwoven layer; providing a meltblown nonwoven comprising bicomponent fibers according to embodiments of the present disclosure 204; placing the nonwoven onto the hydrophilic-treated nonwoven layer; applying hot air 205 to the nonwoven and nonwoven layer; applying a hydrophilic treatment 206 to the nonwoven layer and the nonwoven; running the hydrophilic-treated nonwoven layer and the nonwoven through a hot air oven 207; and using a superabsorbent polymer dosing system 208 to dose the nonwoven layer and the nonwoven with superabsorbent polymer material 209 such that the superabsorbent polymer material is interconnected within the nonwoven layer and nonwoven. An absorbent article according to embodiments herein can result and can then be wound, slit, bonded, and/or combined with other components to form, for example, a diaper.

### Test Methods

### Liquid Absorption Performance—ATUL AND RUL

Acquisition time under load (ATUL) and rewet under load (RUL) are two tests commonly used to evaluate diaper performance. The ATUL test is used to evaluate the urine absorption of a diaper. In general, liquid is applied to a diaper under load and the amount of time it takes for liquid to be absorbed is measured. After repeated insults, the wetness of the absorbing surface is determined in the RUL test. The RUL test in general is a measurement of the amount of fluid the article released under pressure.

For the ATUL test, an example is stretched flat on the examination table over a foam pad to guarantee a flat surface during measurement. The insult point is calculated and marked at 2.5 cm from the example center. A conventional dosing unit is used. The conventional dosing unit includes a plate (10×30 cm) and a cone with a 40 mm diameter for holding and dispensing liquid. The conventional dosing unit also includes two, 4 kg weights (about 27 g/cm² in density).

The conventional dosing unit, along with the two, 4 kg weights, is placed on examples for a time of five minutes. Then, 70 ml of red-dyed deionized water is dispensed from the conventional dosing unit and the amount of time (seconds) that is needed for the example to absorb the liquid such that the liquid disappears from the cone of the dosing unit is recorded as ATUL-1. After five minutes, an additional 70 ml is dispersed and again the amount of time (seconds) that is needed for the example to absorb the liquid is recorded as ATUL-2. This procedure of adding 70 ml and recording the time it takes the example to absorb the liquid is repeated two more times for ATUL-3 and ATUL-4. The ATUL times for the examples are reported in the table below.

After the fourth ATUL test, the RUL at 15 seconds and 120 seconds (2 minutes) intervals is determined. The weight of two clean filter papers is recorded. The filter papers are placed on the left and right side of the liquid addition points and then are covered with the two, 4 kg weights. After fifteen second, the two weights are removed, and the weight of the filter papers is recorded. For RUL at 120 seconds (2 minutes), two filter papers are weighed, and the weight of the papers are measured after 120 seconds (2 minutes) of being under load of the two 4 kg weights. Accordingly, the RUL at 15 seconds is measured in grams and equals the weight of the two filter papers placed on the example for 15 seconds under the load of 2×4 kg weights minus the weight of the two filter papers weighed before the test. The RUL at 120 seconds (2 minutes) equals the weight of the two filter papers placed on the examples for 120 seconds under the load of 2×4 kg weights minus the weight of the two filter papers weighed before the test.

### Extensibility

Extensibility is measured using a tensile test according to ISO 9073-3 with 50×250 mm test specimen of the example at a test speed of 100 mm/min.

### Recyclability

A fraction of the example is cut into small pieces and fed into a mixing chamber. From this, 1 mm thick plaques are compression molded from which dog-bone samples are cut out. The mechanical parameters are tested and measured with the help of a tensile machine in accordance with ISO 527-3 norm, with a 50 mm/min test speed.

### Examples

The following examples illustrate features of the present disclosure but are not intended to limit the scope of the disclosure. The following experiments analyzed the performance of embodiments of the absorbent layer described herein.

The materials for the Comparative Examples include absorbent layers of industry diapers. Comparative Example A is a commercially available Coop Prix size four diapers that comprises an absorbent article including an acquisition distribution layer and cellulose fluff pulp mixed with superabsorbent polymer material. Comparative Example B is a PAMPERS® Baby-Dry™, size 4, commercially available from and marketed by Proctor & Gamble. Comparative Example B comprises an absorbent article having an acquisition distribution layer, cellulose fluff pulp, and a nonwoven including superabsorbent polymer material.

An inventive absorbent article comprising absorbent layers according to one embodiment of the present disclosure is prepared as Inventive Example 1. Inventive Example 1 includes two nonwovens, each nonwoven comprising a plurality of bicomponent fibers. The nonwovens are formed using a bicomponent spunbond pilot line that has two extruders and a single spinneret. One extruder, which is used to form the first region of bicomponent fibers, extrudes a polypropylene, Total PPH 9099 Polypropylene, Homopolymer, commercially available from Total S.A. The other extruder, which is used for the second region of the bicomponent fibers, extrudes a linear low density polyethylene, ASPUNT™ 6834 commercially available from The Dow Chemical Company. Total throughput is kept constant at 180 kg/h, and cabin pressure is maintained at 2300 Pa. Spinneret/die temperature is set at 250° C. A bicomponent spinneret in a side-by-side configuration with 2860 holes at size 0.6 mm is used. Needle punch conditions are as follows: 6,660 needles per meter, stich density of 48 s/cm³, 8 mm space, 2420 strokes/min, and line speed of 33.6 m/min. The plurality of bicomponent fibers are drawn to a nominal denier of 4.2 g/9000 m. Bonding of the web took place between rollers to form the nonwovens. The nonwovens formed from the bicomponent fibers have a basis weight of 83.5 gsm, thickness after needle punch of 18 micron/gsm, and thickness after needle punch and hot air bonding of 30.4 micron/gsm. 11×37 cm sheets of each of the two nonwovens are dipped in a hydrophilic treatment solution (99 wt. % water and 1 wt. % PHP 90), and the nonwovens are permitted to dry. Absorbent layers are prepared by taking the dried hydrophilic-treated nonwovens and dosing 13 grams of superabsorbent polymer material, FAVOR®, commercially available from Evonik, such that superabsorbent polymer materials is interconnected within the nonwovens.

An inventive absorbent article comprising a three-dimensional random loop material according to one embodiment of the present disclosure is prepared as Inventive Example 2. Inventive Example 2 is prepared by taking 11 cm×37 cm×1.5 cm three-dimensional random loop material, spraying it with an adhesive, and dosing it with 13 grams of superabsorbent polymer material, FAVOR®, commercially available from Evonik, such that the superabsorbent polymer material is adhered to the three-dimensional random loop material. An acquisition distribution layer from a PAMPERS® Baby-DryT™, (commercially available from and marketed by Proctor & Gamble) is then placed on top of the three-dimensional random loop material with superabsorbent polymer material to form Inventive Example 2.

To measure the liquid absorption performance under ATUL and RUL tests described above, the absorbent article of Comparative Example A and B were removed from the examples. The removed absorbent article of Comparative Example A is designated as Comparative Example 1 and the removed absorbent article of Comparative Example B is designated as Comparative Example 2.

The liquid absorption performance of Comparative Examples 1 and 2 and Inventive Examples 1 and 2 are measured according to the ATUL and RUL test methods described above and reported in the table below. The time in seconds for the ATUL (at 5 minutes and 70 ml liquid) is recorded as the acquisition time under load 1 (ATUL-1); the next ATUL (at next 5 minutes 70 ml) is recorded as the acquisition time under load 2 (ATUL-2); the next ATUL (at next 5 minutes 70 ml) is recorded as the acquisition time under load 3 (ATUL-3); the next ATUL (at next 5 minutes 70 ml) is recorded as the acquisition time under load 4 (ATUL-4). RUL at 15 seconds is reported as RUL-1, and RUL at 120 seconds is reported as RUL-2, in the table below. A lower ATUL or RUL indicates a better liquid absorption performance. Although, in some aspects, Comparative Example 2 performs better than Inventive Examples 1 and 2, it is noted that Comparative Example 2 includes additional materials-both cellulose fluff pulp and an acquisition distribution layer.

TABLE 1
ATUL AND RUL

| | ATUL- | ATUL- | ATUL- | ATUL- | RUL- | RUL- |
|---|---|---|---|---|---|---|
| | 1 | 2 | 3 | 4 | 1 | 2 |
| | (s) | (s) | (s) | (s) | (g) | (g) |
| Comparative | 45 | 350 | 730 | 720 | 0.24 | 0.39 |
| Example 1 | | | | | | |
| Comparative | 12 | 32 | 34 | 35 | 0.08 | 0.37 |
| Example 2 | | | | | | |
| Inventive | 26 | 74 | 72 | 72 | 0.16 | 0.22 |
| Example 1 | | | | | | |
| Inventive | 3 | 96 | 150 | 191 | 0.32 | 0.72 |
| Example 2 | | | | | | |

The extensibility of Comparative Example 2 and Inventive Examples 1 and 2 are measured according to the test method described above. The extensibility of Comparative Example 1 is not reported below, as the example fell apart and was not able to withstand the testing for recording of an accurate measurement. As shown in the table, the extensibility of Inventive Examples 1 and 2 is significantly improved in comparison to Comparative Example B.

TABLE 2
Extensibility

| Example No. | Extensibility (%) |
|---|---|
| Comparative Example B | 28 |
| Inventive Example 1 | 105 |
| Inventive Example 2 | 164 |

For recyclability, as described in the test method above, a plastic fraction of Comparative Example 2 was separated from the cellulose pulp and the SAP, and the plastic fraction of the Comparative Example 2 and Inventive Example 1 were cut into small pieces and fed into a mixing chamber and processed. From the samples mechanical parameters were measured with the help of a tensile machine, as outline in the test method above.

The table below shows the elongation at break (%) and stress at break (Mpa) for Comparative Example B and Inventive Example 1. These test measurements relate to the recyclability of the materials, and the measurements demonstrate an improvement in recyclability of Inventive Example 1 in comparison to Comparative Example 2.

TABLE 3
Recyclability

| | Comparative | Inventive |
|---|---|---|
| | Example 2 | Example 1 |
| Elongation at break (%) | 3 | 7 |
| Stress at break (Mpa) | 11.74 | 19.16 |

Every document cited herein, if any, including any cross-referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An absorbent layer suitable for use in an absorbent article, the absorbent layer comprising:
a nonwoven comprising a plurality of bicomponent fibers, wherein each bicomponent fiber has a first region and a second region,
wherein the weight ratio of the first region to the second region is at least 10/90 and not more than 90/10,
wherein the first region comprises a first polymer composition in an amount of at least 75 wt. % based on total weight of the first region and the second region comprises a second polymer composition in an amount of at least 75 wt. % based on total weight of the second region; and
a superabsorbent polymer material interconnected within the nonwoven.

2. The absorbent layer of claim 1, wherein the nonwoven is a hydrophilic-treated nonwoven.

3. The absorbent layer of claim 1, wherein the weight ratio of nonwoven to superabsorbent polymer material is at least 20/80 and not more than 80/20.

4. The absorbent layer of claim 1, wherein the first region comprises one or more of the following: a polyethylene, a polypropylene, a polyolefin elastomer, and a polyethylene terephthalate.

5. The absorbent layer of claim 1, wherein the second region comprises one or more of the following: a polyethylene, a polypropylene, a polyolefin elastomer, and a polyethylene terephthalate.

6. The absorbent layer of claim 1, wherein the first region is a core region of the bicomponent fiber and the second region is a sheath region of the bicomponent fiber, where the sheath region surrounds the core region.

7. The absorbent layer of claim 1, wherein each bicomponent fiber has a fiber centroid, and the first region has a first fiber centroid and the second region has a second fiber centroid, and wherein the regions are arranged such that the first fiber centroid and the second fiber centroid is not the same as the fiber centroid.

8. An absorbent article comprising the absorbent layer of claim 1, wherein the absorbent article is free from an acquisition distribution layer or cellulose pulp.

9. A method for making an absorbent layer suitable for use in an absorbent article comprising:
providing a nonwoven comprising a plurality of bicomponent fibers, wherein each bicomponent fiber has a first region and a second region; wherein the weight ratio of the first region to the second region is at least 10/90 and is not more than 90/10; wherein the first region comprises a first polymer composition in an amount of at least 75 wt. % based on total weight of the first region and the second region comprises a second polymer composition in an amount of at least 75 wt. % based on total weight of the second region; and
dosing superabsorbent polymer material on the nonwoven such that the superabsorbent polymer material is interconnected within the nonwoven.

10. An absorbent layer suitable for use in an absorbent article, the absorbent layer comprising:
a three-dimensional random loop material; and
a superabsorbent polymer material adhered to the three-dimensional random loop material.
